(19) United States
(12) Patent Application Publication
(10) Pub. No.: US 2016/0002159 A1
(43) Pub. Date: Jan. 7, 2016

(54) PERFLUORO-TERT-BUTYL HYDROXYPROLINE

(71) Applicants: Neal ZONDLO, Newark, DE (US); Anil PANDEY, Newark, DE (US); Caitlin TRESSLER, Newark, DE (US)

(72) Inventors: Neal ZONDLO, Newark, DE (US); Anil PANDEY, Newark, DE (US); Caitlin TRESSLER, Newark, DE (US)

(21) Appl. No.: 14/767,866

(22) PCT Filed: Feb. 12, 2014
(86) PCT No.: PCT/US14/16121

§ 371 Date: Aug. 13, 2015

Related U.S. Application Data
(60) Provisional application No. 61/764,190, filed on Feb. 13, 2013.

(51) Int. Cl. C07D 207/16; G01N 33/58; A61K 49/10

(52) U.S. Cl. CPC C07D 207/16; A61K 49/10; G01N 33/58; G01N 2458/00; G01N 2440/14; G01N 2333/912

(57) ABSTRACT

The present invention provides novel analogues of alpha amino acids, comprising a perfluoro-tert-butyl group, and molecules comprising the novel analogues. Also provided are a wide range of applications of the novel analogues in therapeutics, theranostics and pharmaceuticals as well as in imaging applications. In particular, the use of the novel analogues in detecting or modifying a target molecule is provided.

### CROSS-REFERENCE TO RELATED APPLICATION

This application claims the benefit of U.S. Provisional Application No. 61/764,190, filed Feb. 13, 2013, the contents of which are incorporated herein by reference in their entireties for all purposes.

### REFERENCE TO U.S. GOVERNMENT SUPPORT

This work is supported by grants from the National Science Foundation (Grant Number CHE-0547973) and National Institute of Health (Grant Number GM093225). The United States has certain rights in the invention.

### FIELD OF THE INVENTION

The invention relates generally to analogues of amino acids and their uses in therapeutics, theranostics and pharmaceuticals as well as in imaging applications.

### BACKGROUND OF THE INVENTION

Fluorine is an atom with unique properties. Fluorine is the most electronegative atom, and fluorine incorporation in a molecule increases its hydrophobicity. The unique chemical properties of fluorine have led to its wide incorporation in pharmaceuticals and other biologically active compounds. The most abundant nucleus of fluorine, ¹⁹F, also is a spin-1/2 nucleus, and thus may be actively detected in imaging using magnetic resonance techniques, including nuclear magnetic resonance (NMR), magnetic resonance spectroscopy (MRS), and magnetic resonance imaging (MRI).

Currently, MRI typically does not provide specific information about disease, because MRI is based on detection of differences in water in diseased versus healthy tissue. Development of new probes to detect specific molecular events associated with disease would substantially increase the information content of MRI. ¹⁹F imaging has enormous potential because of its specificity (high signal to noise due to the absence of fluorine in vivo; application to detect specific molecular events), its high sensitivity compared to proton (similar sensitivity), and its application using commercial proton magnetic resonance instruments. The potential of ¹⁹F magnetic imaging in medicine is currently substantially limited by a need to achieve increased sensitivity for applications. An ideal approach to enhance specificity and sensitivity of ¹⁹F magnetic resonance imaging and magnetic resonance spectroscopy would involve the incorporation of an intense fluorine signal in native ligands in a manner that is minimally disruptive of structure.

Magnetic resonance imaging (MRI) is a widely utilized technique for biomedical imaging. The particular advantages of MRI include safety (the absence of radioactive molecules) and practicality (MRI is based on detection of ubiquitous ¹H nuclei). ¹⁹F is a nucleus with 100% natural abundance and sensitivity close to that of ¹H. In addition, most ¹H probes (instruments) can be readily adapted to detect ¹⁹F. Thus, in theory, ¹⁹F MRI can be achieved readily with currently available instrumentation. A particular advantage of magnetic imaging using ¹⁹F is the signal specificity (that is, the detection of specific molecular events based on labeling of specific molecules with ¹⁹F probes) and the absence of background signals, in contrast to ¹H imaging, where water is the dominant signal and water relaxation the dominant mode of imaging. Thus, ¹⁹F MRI has substantial potential for the imaging of specific processes in vivo, including intracellular and extracellular changes indicative of disease. However, the potential of ¹⁹F magnetic imaging is currently limited by the need for improved approaches to increase the signal to noise for ¹⁹F signals. In part, signal to noise may be increased via the incorporation of modern probe technology on MRI instrumentation. However, despite the ability of modern NMR probes to detect ¹⁹F signals at low μM concentration, an improvement in signal to noise is also necessary due to imaging constraints.

¹⁹F magnetic resonance spectroscopy (MRS) has enormous potential because of its specificity, its high sensitivity comparable to proton, its large chemical shift dispersion, and its application using commercial proton magnetic resonance imaging instruments. The potential of ¹⁹F magnetic imaging in medicine is currently substantially limited by a need to achieve increased sensitivity for applications. One approach to increase signal to noise is via molecules with multiple degenerate fluorines yielding a singlet signal. This approach is a well-demonstrated method to allow observation of ¹⁹F signals on standard instrumentation. Low signal to noise is observed in molecules with single fluorine substitution, with trifluoromethyl groups commonly employed to increase signal-to-noise at least 3-fold (due to the homotopic nature of the fluorines, they appear as a single signal of 3-fold greater intensity). To date, these approaches to increase signal to noise have typically employed nonspecific fluorocarbon contrast agents. As contrast agents, however, they do not report directly on cellular state. However, approaches that include specific biomolecules that have been modified to incorporate fluorine signals hold the potential to provide a direct readout of biological activity in a manner than can provide a detailed readout of cellular state.

One particularly exciting possibility with ¹⁹F imaging that is not easily achieved with ¹H imaging is the ability to detect specific enzymatic activity directly. Pioneering work by Dalvit and coworkers has demonstrated, in simple solution experiments, that labeling with simple trifluoromethyl labels (e.g. N-terminal peptide labeling as the trifluoroacetamide) results in peptide labeling with ¹⁹F that allows readout, by simple ¹⁹F NMR spectroscopy via chemical shift changes, the detection of either phosphorylation by kinase or cleavage of a peptide by protease. These techniques demonstrated the potential of ¹⁹F NMR for detection of kinase or protease activity, but were limited by modest sensitivity, due to both limited methods of labeling that did not involve similar substitution at a canonical amino acid and the presence of a limited number of equivalent fluorines. However, they provide critical proof of principle, because even with a simple design they could be used to detect activity in solution. The development of aggregates for ¹⁹F imaging is also promising, and has been specifically applied by Hamachi to the solution detection of protease activity.

The potential of specific ¹⁹F MRS for imaging has also been exploited for the detection of proteins in vivo. While this has typically been done using small molecule agents that bind to proteins, as in the case of ¹⁹F imaging of amyloid plaques, ¹⁹F NMR has also been used to monitor protein expression and protein folding in living E. coli cells. There remains a need for highly sensitive biological probes or tracers useful for medicinal chemistry, including the identification of intracellular and extracellular events in solution, in extracts, in cells, in tissue, and in vivo. The perfluoro-tert-butyl group provides the specific advantage of nine magnetically equivalent fluorine atoms in a molecule, which are not coupled to other atoms, greatly increasing the sensitivity in detection due to the presence of a single peak of high intensity from the perfluoro-tert-butyl group. This invention describes the incorporation of the perfluoro-tert-butyl group in amino acids, which are constituents in small molecules and pharmaceuticals, peptides, proteins, and materials.

### SUMMARY OF THE INVENTION

The present invention relates to novel analogues of amino acids and their uses.

According to a first aspect of the present invention, an analogue of an alpha amino acid is provided. The analogue comprises a perfluoro-tert-butyl group. The analogue may be perfluoro-tert-butyl hydroxyproline, perfluoro-tert-butylalanine, perfluoro-tert-butyl homoserine, perfluoro-tert-butyl glycine, perfluoro-tert-butyl aspartate, perfluoro-tert-butyl glutamate or perfluoro-tert-butyl tyrosine. Preferably, the analogue is perfluoro-tert-butyl hydroxyproline (Hyp). The Hyp may be 2S,4R (Hyp), 2S,4S (Hyp), 2R,4R (Hyp) or 2R,4R (Hyp). The analogue may be Fmoc-perfluoro-tert-butyl hydroxyproline or Boc-perfluoro-tert-butyl hydroxyproline.

According to a second aspect of the present invention, a molecule comprising the analogue of the present invention is provided. A composition comprising the molecule is also provided.

According to a third aspect of the present invention, a method for diagnosing, treating or preventing a disease or condition in a subject in need thereof is provided. The method comprises administrating to the subject an effective amount of the composition of the present invention.

According to a fourth aspect of the invention, a method for detecting a target molecule in a sample is also provided. The detection method comprises exposing the sample to an effective amount of a test molecule comprising an analogue of the present invention. The analogue interacts with the target molecule. The method further comprises detecting the interaction. The presence of the interaction indicates the presence of the target molecule in the sample. The method may further comprise quantifying the target molecule in the sample.

Where the target molecule is modified upon exposure, the detection method may further comprise detecting the modified target molecule. The modification of the target molecule indicates the presence of the interaction. The affinity between the target molecule and a subject molecule in the sample may be altered upon exposure.

Where the test molecule is modified upon exposure, the detection method may further comprise detecting the modified test molecule. The modification of the test molecule indicates the presence of the interaction.

Where the test molecule binds a biological molecule in the sample upon exposure, the detection method may further comprise detecting the test molecule bound to the biological molecule. The presence of the test molecule bound to the biological molecule indicates the presence of the interaction.

Where the test molecule binds a cell in the sample upon exposure, the detection method may further comprise detecting the test molecule bound to the biological molecule. The presence of the test molecule bound to the cell indicates the presence of the interaction.

In the detection method, the test molecule may be detected by ¹⁹F NMR spectroscopy, magnetic resonance stimulation (MRS) or magnetic resonance imaging (MRI), preferably by magnetic resonance imaging (MRI) in vivo.

The sample used in the detection method may be obtained from a subject or in a subject, and the presence of the target molecule in the sample may indicate that the subject suffers or is predisposed to a disease or condition associated with the target molecule. The detection method may further comprise treating or preventing the disease or condition in the subject.

According to a fifth aspect of the present invention, a method for modifying a target molecule in a sample is provided. The modification method comprises exposing the sample to an effective amount of a test molecule comprising an analogue of the present invention.

### BRIEF DESCRIPTION OF THE DRAWINGS

FIG. 1 illustrates novel perfluoro-tert-butyl amino acid analogues. The 9 fluorines in each perfluoro-tert-butyl group are chemically equivalent, resulting in a sharp singlet by NMR and high signal to noise. As amino acid analogues, they can be incorporated internally at native sites within peptides and proteins near functional sites, rather than as external labels, maximizing the NMR response.

FIG. 2 shows (A) ¹H NMR spectrum of peptide Ac-TYP(4R—(OH))N—NH₂ (1) (TYHypN) (SEQ ID NO: 1): amide region; and (B) full ¹H NMR spectrum of peptide Ac-TYHypN—NH₂ (1).

FIG. 3 shows (A) ¹H NMR spectrum of peptide Ac-TYP(4S—OH)N—NH2 (4) (TYhypN—NH₂) (SEQ ID NO: 2): amide region; and (B) full ¹H NMR spectrum of peptide Ac-TYhypN—NIH₂ (4).

FIG. 4 shows (A) ¹H NMR spectrum of peptide Ac-TYP(4R—OC(CF₃)₃)N—NH₂ (55) (SEQ ID NO: 3): amide region; (B) full ¹H NMR spectrum of peptide Ac-TYP(4R—OC(CF₃)₃)N—NH₂ (55); (C) ¹⁹F NMR spectrum of peptide Ac-TYP(4R—OC(CF₃)₃)N—NH₂ (55); and (D) Crude HPLC chromatogram, top: Crude HPLC chromatogram of peptide Ac-TYhypN—NH₂ (4), and bottom: Crude HPLC chromatogram of peptide Ac-TYP(4R—OC(CF₃)₃)N—NH₂(55).

FIG. 5 shows (A) (A) ¹H NMR spectrum of peptide Ac-TYP(4S—OC(CF₃)₃)N—NH₂ (56) (SEQ ID NO: 4): amide region; (B) Full ¹H NMR spectrum of peptide Ac-TYP(4S—OC(CF₃)₃)N—NH₂ (56); (C) ¹⁹F NMR spectrum of peptide Ac-TYP(4S—OC(CF₃)₃)N—NH₂ (56); and (D) Crude HPLC chromatogram (top: Crude HPLC chromatogram of peptide Ac-TYHypN—NH₂ (1); bottom: Crude HPLC chromatogram of peptide Ac-TYP(4S—OC(CF₃)₃)N—NH₂(56)).

FIG. 6 shows ¹⁹F NMR spectra of phosphorylation of peptides containing 4R- and 4S-perfluoro-tert-butyl hydroxyproline by protein kinase A at to and t=5 minutes (top two panels) and Akt/PKB at t₀ and t=1 h (bottom two panels), to =spectrum of the non-phosphorylated peptide prior to addition of kinase. The peptides are Ac-LRR4R-Hyp(C₄F₉)SLGAK-NIH₂ (SEQ ID NO: 5), Ac-LRR4S-hyp(C₄F₉)SLGAK-NH₂ (SEQ ID NO: 6), Ac-AKRARERT4R-Hyp(C₄F₉)SFGHHA-NH₂ (SEQ ID NO: 7) and Ac-AKRARERT4S-hyp(C₄F₉)SFGHHA-NH₂ (SEQ ID NO: 8). Minor unlabeled peaks correspond to expected small populations of cis proline amide bond. Extent of phosphorylation observed by ¹⁹F NMR was confirmed by HPLC and ESI-MS.

FIG. 7 shows real time detection of PKA activity (top) and PKA inhibition by H-89 (bottom) in HeLa extracts by ¹⁹F NMR on peptide with 4R-perfluoro-tert-butyl hydroxyproline. NMR experiments were conducted on a single sample at 5 minute time increments.

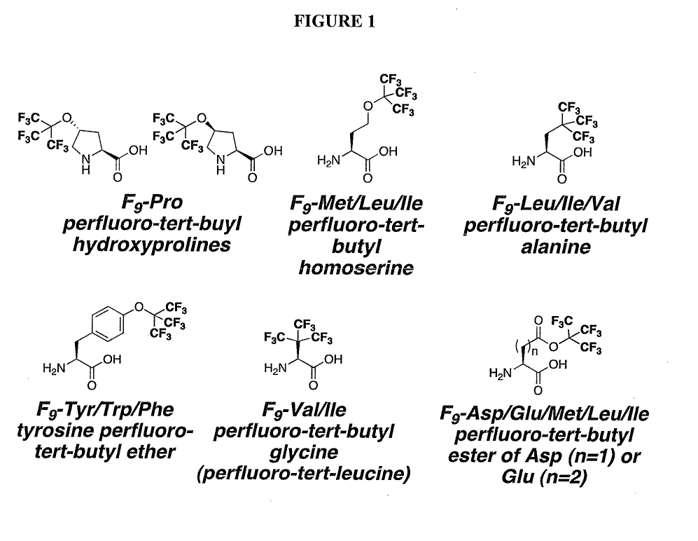

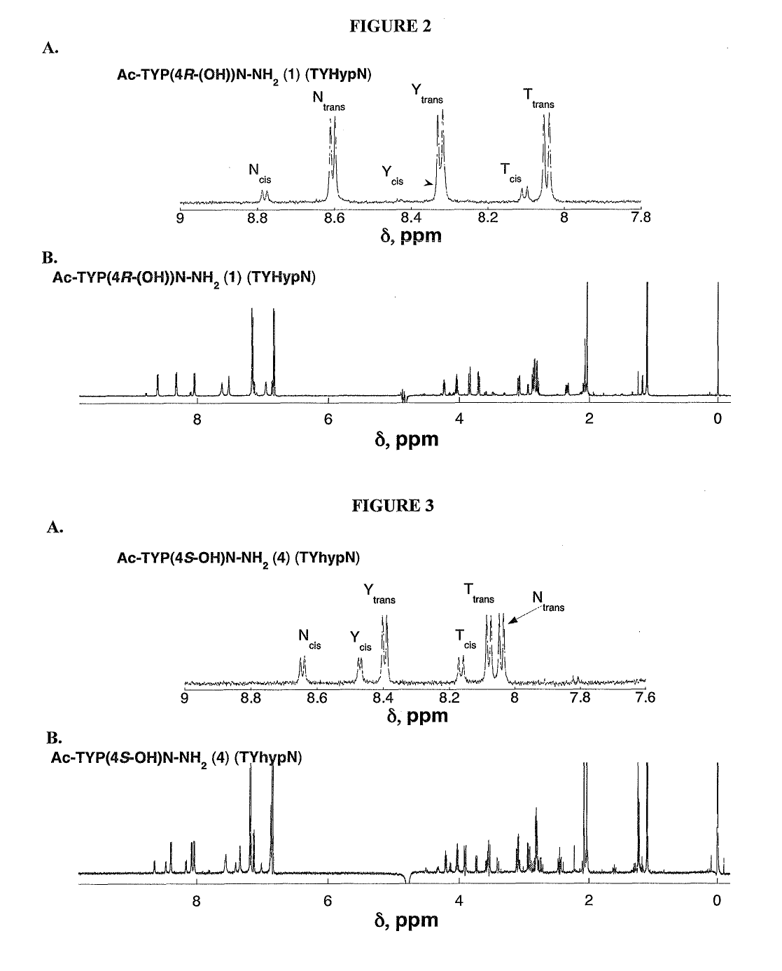

### DETAILED DESCRIPTION OF THE INVENTION

The present invention relates to novel analogues of amino acids and their uses. In particular, these novel amino acid analogues may be incorporated in various molecules, including small molecules, peptides, proteins, and other polymers, that are useful for applications in therapeutics, theranostics and pharmaceuticals as well as in imaging applications.

The present invention is based on the discovery of methods for incorporating perfluoro-tert-butyl groups, specifically perfluoro-tert-butyl hydroxyproline, into small molecules, peptides, and proteins as novel amino acids. Perfluoro-tert-butyl groups have 9 equivalent fluorines, and thus have a 9-fold increase in signal-to-noise over single fluorines. At least as importantly, perfluoro-tert-butyl groups are sharp singlets by NMR, further increasing signal-to-noise and operational simplicity, meaning that most existing proton-based instrumentation can readily be adjusted to detect peptides containing perfluoro-tert-butyl groups. Because of high signal to noise and the ability to be incorporated within small molecules, peptides and proteins, perfluoro-tert-butyl hydroxyproline has broad potential applications in magnetic imaging (NMR, MRS, MRI), both in vitro and in vivo. Tert-butyl groups also have broad importance in medicinal chemistry due to their hydrophobicity and symmetry, leading to enhanced binding to targets. The amino acid tert-leucine (also known as tert-butyl glycine) is employed in a number of FDA-approved pharmaceuticals. Fluorination is also a broadly employed strategy in medicinal chemistry to enhance affinity and stability to pharmaceuticals. Perfluoro-tert-butyl hydroxyproline thus could also be used in medicinal chemistry.

Novel analogues of amino acids are provided. The term “amino acid” used herein refers to a standard amino acid that is naturally incorporated into a peptide. The amino acid is preferably an alpha amino acid, which contains an amino group and a carboxylic acid group that are separated by one carbon. An alpha amino acid may have a hydrophobic nonacidic side chain (e.g., glycine (Gly), alanine (Ala), valine (Val), leucine (Leu), isoleucine (Ile), proline (Pro), tryptophan (Trp), phenylalanine (Phe) and methionine (Met)), a hydrophobic acidic side chain (e.g., cysteine (Cys) and tyrosine (Tyr)), a hydrophilic nonacidic side chain (e.g., serine (Ser), threonine (Thr), asparagine (Asn) and glutamine (Gln)), a hydrophilic acidic side chain (e.g., aspartic acid (Asp) and glutamic acid (Glu)), or a hydrophilic basic side chain (e.g., lysine (Lys), arginine (Arg) and histidine (His)). Preferably, the alpha amino acid is selected from the group consisting of Pro, Leu, Met, Gly, Ile, Val, Phe, Tyr, Trp, Asp and Glu. More preferably, the alpha amino acid is selected from the group consisting of Pro, Leu, Met, Gly, Asp, Glu and Tyr. Most preferably, the alpha amino acid is Pro.

The term “analogue of an amino acid” used herein refers to a derivative of an amino acid that may be incorporated into a molecule (e.g., a peptide, protein, polymer or small molecule) in place of the amino acid. A derivative of an amino acid is a molecule derived from the amino acid via one or more chemical reactions, biological reactions or a combination thereof. The derivative may be an amide, carbamate (e.g., Fmoc, Boc, Cbz protected), free acid, amide, ether, ester or alcohol.

The analogue may have a perfluoro-tert-butyl group. For example, the analogue may be perfluoro-tert-butyl hydroxyproline, perfluoro-tert-butylalanine, perfluoro-tert-butyl homoserine, perfluoro-tert-butyl glycine, perfluoro-tert-butyl aspartate, perfluoro-tert-butyl glutamate or perfluoro-tert-butyl tyrosine. Preferably, the analogue is perfluoro-tert-butyl hydroxyproline, perfluoro-tert-butylalanine or perfluoro-tert-butyl homoserine. More preferably, the analogue is perfluoro-tert-butyl hydroxyproline (Hyp). The Hyp may be one of the four stereoisomers, 2S,4R (Hyp), 2S,4S (Hyp), 2R,4R (Hyp) and 2R,4R (Hyp).

The analogue may also have a functional group such as a fluorenylmethyloxycarbonyl group (Fmoc) or a butyl dicarbonyl group (Boc). For example, the analogue may be Fmoc-perfluoro-tert-butyl hydroxyproline or Boc-perfluoro-tert-butyl hydroxyproline.

The amino acid analogues may be prepared by chemical synthesis, biological synthesis, or a combination of both. For example, stereospecifically modified proline residues may be prepared by peptide synthesis. Peptides may be synthesized by standard solid-phase-peptide-synthesis to incorporate Fmoc-Hydroxyproline (4R-Hyp). In an automated manner, the Hyp hydroxyl is protected and the remainder of the peptide synthesized. After peptide synthesis, the Hyp protecting group is orthogonally removed and Hyp selectively modified to generate substituted proline amino acids, with the peptide main chain functioning to “protect” the proline amino and carboxyl groups. The proline derivatives may be prepared without solution phase synthesis.

A molecule comprising the amino acid analogue of the present invention is provided. The molecule may be a small molecule, a peptide, a protein, or another polymer. The molecule may be selected from the group consisting of a therapeutic agent, a theranostic agent, a pharmaceutical agent, a diagnostic agent and an imaging agent.

The term “small molecule” used herein refers to a molecule of low molecular weight, for example, less than 2000 Daltons. The small molecule may be an organic compound having a biological activity. For examples, the small molecule may be an agent useful for diagnosing, treating or preventing a disease or condition.

The term “peptide” used herein refers to a polymer of amino acid residues with no limitation with respect to the minimum length of the polymer. For example, the peptide may have at least 3, 4, 5, 10, 20, 50 or more amino acid residues. The term “protein” used herein refers one or more peptides having a biological activity. Preferably, the protein comprises a peptide having at least 20 amino acids. The term “peptide” may include a peptide conjugated to a molecule that is not a peptide. The definitions of“peptide” and “protein” include both the full-length and fragments of the peptide or protein, as well as modifications thereof (e.g., glycosylation, phosphorylation, deletions, additions and substitutions).

The amino acid analogues or amino acid analogue containing molecules of the present invention may be synthesized by using conventional techniques. For example, a peptide may be synthesized chemically or biologically using an amino acid analogue in place of, or in excess to, the amino acid. A small molecule containing molecules of the present invention may be prepared by chemical synthesis. A protein may be synthesized chemically or via protein expression using an amino acid analogue in place of, or in excess to, the amino acid. The synthesized analogue or molecules may be analyzed by NMR to identify the effect of substitution on small molecule, peptide, protein, or polymer structure or function.

A method for synthesizing perfluoro-tert-butyl hydroxyproline is provided. The method comprises synthesizing a peptide to incorporate Fmoc-Hydroxyproline (4R-Hyp), protecting the Hyp hydroxyl, synthesizing the reminder of the peptide, removing the Hyp protecting group orthogonally, and selectively modifying Hyp to generate perfluoro-tert-butyl hydroxyproline. The synthesis method may further comprise incorporating the perfluoro-tert-butyl hydroxyproline into a peptide.

A composition comprising the amino acid analogue or the amino acid analogue containing molecule of the present invention is provided. The composition may further comprise a pharmaceutically acceptable carrier or diluent. The composition may further comprise a therapeutic agent, a theranostic agent, a pharmaceutical agent, a diagnostic agent or an imaging agent. The composition of the present invention may have a wide range of applications, including therapeutics, theranostics and pharmaceuticals as well as imaging applications. In particular, the composition may be used to diagnose, treat or prevent a disease or condition.

A medicament comprising an effective amount of the amino acid analogue or the amino acid analogue containing molecule of the present invention is provided. The medicament is useful for diagnosing, treating or preventing a disease or condition in a subject. For each medicament, a preparation method is provided. The preparation method comprises admixing the analogue or the molecule with a pharmaceutically acceptable carrier or diluent.

A method for detecting a target molecule in a sample is provided. The method comprises exposing the sample to an effective amount of a test molecule comprising the amino acid analogue of the present invention. The amino acid analogue interacts with the target molecule. The method further comprises detecting the interaction between the target molecule and the test molecule. The presence of the interaction indicates the presence of the target molecule in the sample. The test molecule may be provided for delivery in a composition at a concentration from about 100 picomolar to about 500 millimolar, preferably from about 10 nanomolar to about 100 micromolar. The test molecule may also be used as pure material. The method may further comprise quantifying the target molecule in the sample.

The target molecule may be selected from the group consisting of a peptide, a protein, a small molecule and a polymer. For example, the target molecule may be an enzyme, such as a protein kinase or a protease. The target molecule may be a therapeutic agent, a theranostic agent, a pharmaceutical agent, a diagnostic agent or an imaging agent.

Where the target molecule is modified upon exposure, the detection method may further comprise detecting the modified target molecule. The modification of the target molecule indicates the presence of the interaction between the target molecule and the test molecule, and therefore the presence of the target molecule. The target molecule may be modified by the test molecule, directly or indirectly. Where the target molecule is an enzyme, the enzymatic activity of the target molecule may be modified upon exposure. The enzymatic activity may be enhanced or inhibited. The test molecule may be an activator, inhibitor, substrate, or ligand of the enzyme. The enzyme may be a protein kinase, a protein phosphatase, or a protease.

Where the target molecule binds a subject molecule in the sample, the affinity between the target molecule and the subject molecule may be altered upon exposure. The affinity may be increased or decreased. In some embodiments, the target molecule may bind to the subject molecule after binding to a ligand, and the modification of the target molecule upon exposure may alter the affinity between the target molecule and the subject molecule, and may thereby regulate the biological activity of the subject molecule. The affinity may be increased or decreased. The target molecule may be selected from the group consisting of estrogen receptor, androgen receptor and p53-MDM2.

Where the test molecule is modified upon exposure, the detection method of the present invention may further comprise detecting the modified test molecule. The modification of the test molecule indicates the presence of the interaction between the target molecule and the test molecule, and therefore the presence of the target molecule. The test molecule may be modified by the target molecule, directly or indirectly.

Where the target molecule is an enzyme that modifies the test molecule, the modification of the test molecule indicates the enzymatic activity of the target molecule. The target molecule may be a protein kinase or protease. For example, the target molecule may be a protein kinase that phosphorylates the test molecule, and phosphorylation of the test molecule indicates the kinase activity of the target molecule. The detection method may further comprise quantifying the enzymatic activity of the target molecule.

Where the test molecule binds a biological molecule in the sample upon exposure, the detection method may comprise detecting the test molecule bound to the biological molecule. The presence of the test molecule bound to the biological molecule indicates the presence of the interaction between the target molecule and the test molecule. The biological molecule may be a protein, carbohydrate, DNA or RNA.

Where the test molecule binds a cell in the sample upon exposure, the detection method may comprise detecting the test molecule bound to the biological molecule. The presence of the test molecule bound to the cell indicates the presence of the interaction between the target molecule and the test molecule. The cell may be a diseased cell, preferably a cancer or tumor cell.

The test molecule may be detected by any conventional physical, chemical or biological method. Preferably, the test molecule is detected by ¹⁹F NMR spectroscopy, magnetic resonance stimulation (MRS) or magnetic resonance imaging (MRI). More preferably, the test molecule is detected by magnetic resonance imaging (MRI) in vivo.

The sample in the detection method of the present invention may comprise a solution, cell extract or living cells. The sample may be obtained from a subject or in a subject, and the presence of the target molecule in the sample indicates that the subject suffers or is predisposed to a disease or condition associated with the target molecule. The disease or condition may be any disease or condition, for example, HIV or HCV infection. The detection method may further comprise treating or preventing the disease or condition in the subject.

For each detection method, a medicament is provided. The medicament comprises an effective amount of the test molecule useful for detecting the target molecule. For each medicament, a preparation method is also provided. The preparation method comprises admixing the test molecule with a pharmaceutically acceptable carrier or diluent.

A method for modifying a target molecule in a sample is provided. The method comprises exposing the sample to an effective amount of a test molecule comprising the amino acid analogue of the present invention. The test molecule may be in an amount from nanograms to grams, preferably from about 1 microgram to about 1000 milligrams.

In the modification method of the present invention, the target molecule may be selected from the group consisting of a therapeutic agent, a theranostic agent, a pharmaceutical agent, a diagnostic agent and an imaging agent. The target molecule may be modified by the test molecule. Where the target molecule is an enzyme, for example, a protein kinase or a protease, the enzymatic activity of the target molecule may be modified upon exposure. The enzymatic activity may be enhanced or inhibited. The test molecule may be an activator or inhibitor of the enzyme.

Where the target molecule binds a subject molecule in the sample, the affinity between the target molecule and the subject molecule may be altered upon exposure. The affinity may be increased or decreased. In some embodiments, the target molecule may bind the subject molecule after binding to a ligand, and the modification of the target molecule upon exposure may alter the affinity between the target molecule and the subject molecule, thereby regulating the biological activity of the subject molecule. The affinity may be increased or decreased. The target molecule may be selected from the group consisting of estrogen receptor, androgen receptor and MDM2.

The sample in the modification method of the present invention may comprise a solution, cell extract or living cells. The sample may be obtained from a subject or in a subject, and the presence of the target molecule in the sample indicates that the subject suffers or is predisposed to a disease or condition associated with the target molecule. The disease or condition may be any disease or condition, for example, HIV or HCV infection. The modification of the target molecule may result in treatment or prevention of the disease or condition in the subject.

For each modification method, a medicament is provided. The medicament comprises an effective amount of the test molecule useful for modifying the target molecule. For each medicament, a preparation method is also provided. The preparation method comprises admixing the test molecule with a pharmaceutically acceptable carrier or diluent.

Perfluoro-tert-butyl hydroxyproline (Hyp) may be synthesized and incorporated into peptides. Stereoisomers may be synthesized, and each may have different physical properties.

As a high signal-to-noise ligand for ¹⁹F magnetic imaging (NMR, MRS, and MRI), perfluoro-tert-butyl hydroxyproline is suitable for applications in solution, in cells, or in vivo. Having its 9 equivalent fluorines that are not coupled to one another, it has a signal as a singlet (sharp peak) with no coupling (splitting) and 9 times greater than a single fluorine (and more than that in practice, since single fluorines typically have their signal reduced due to coupling).

Molecules containing perfluoro-tert-butyl hydroxyproline can be rapidly detected using NMR at nanomolar concentrations and used as probes of enzymatic processes in solution and in cell extracts. Peptides containing these amino acids may be used to probe intracellular processes by NMR.

In addition, perfluoro-tert-butyl hydroxyproline may substitute for native amino acids in peptides (e.g., at Pro, Leu, Ile). Peptides containing this amino acid are readily recognized by protein kinases, yielding phosphorylated peptides with the phosphorylation site immediately adjacent to the perfluoro-tert-butyl hydroxyproline. Thus, these amino acids may be recognized by native enzymes and native proteins, and thus can directly be used as probes of native processes (for example, using ¹⁹F NMR to detect protein kinase activity in cells). This amino acid may be used in a broad range of potential applications, for example, imaging, including in cell and in vivo imaging in ways that provide far greater and far more specific diagnostic information than traditional MRI, which uses water.

Fluorinated amino acids are more hydrophobic than analogous non-fluorinated amino acids. The incorporation of fluorines in pharmaceuticals is a widely recognized strategy to increase potency. There are numerous FDA-approved drugs containing prolines (or, by analogy with the above, large hydrophobic amino acids like leucine or isoleucine or larger nonnatural amino acids), and biologically active molecules containing fluorine are expected in many cases to be more potent, so this amino acid could have wide application for substitution in molecules for pharmaceutical screening and drug approval.

Perfluoro-tert-butyl hydroxyproline provides a sensitive, specific probe of peptide or protein function, including localization and modification, with specific potential application to imaging in vitro, in living cells, in tissue, and in vivo, using NMR and MRI spectroscopies. For example, it may be used in a novel approach to introduce ¹⁹F atoms into peptides and proteins in a minimally disruptive way.

Perfluoro-tert-butyl hydroxyproline may be used to make molecules that not only have significant biological potency but also function as highly sensitive biological probes or tracers by interacting with native proteins. For example, these molecules may be applied within the ligands to the estrogen receptor to develop methods to simultaneously image and inhibit the estrogen receptor with a novel highly potent ligand containing perfluoro-tert-butyl hydroxyproline. Further, perfluoro-tert-butyl hydroxyproline may be incorporated into pharmaceuticals.

¹⁹F-labeled peptides (conjugated to cell-penetrating peptides) may be used in an approach to identify protein kinase activity in living cancer cells, with subsequent application of ¹⁹F imaging to interrogate the cellular changes in cancers and other diseases in vivo. Perfluoro-tert-butyl hydroxyproline could also be employed to understand distribution and localization of proteins, hormones, and pharmaceuticals.

The term “about” as used herein, when referring to a measurable value such as an amount, a percentage, and the like, is meant to encompass variations of ±20%, ±10%, more preferably ±5%, even more preferably ±1%, and still more preferably ±0.1% from the specified value, as such variations are appropriate.

### Example 1

### Amino Acid Analogue Synthetic Approaches

### A. Synthesis of Fmoc-2S,4R-perfluoro-tert-butyl-hydroxyproline (7)

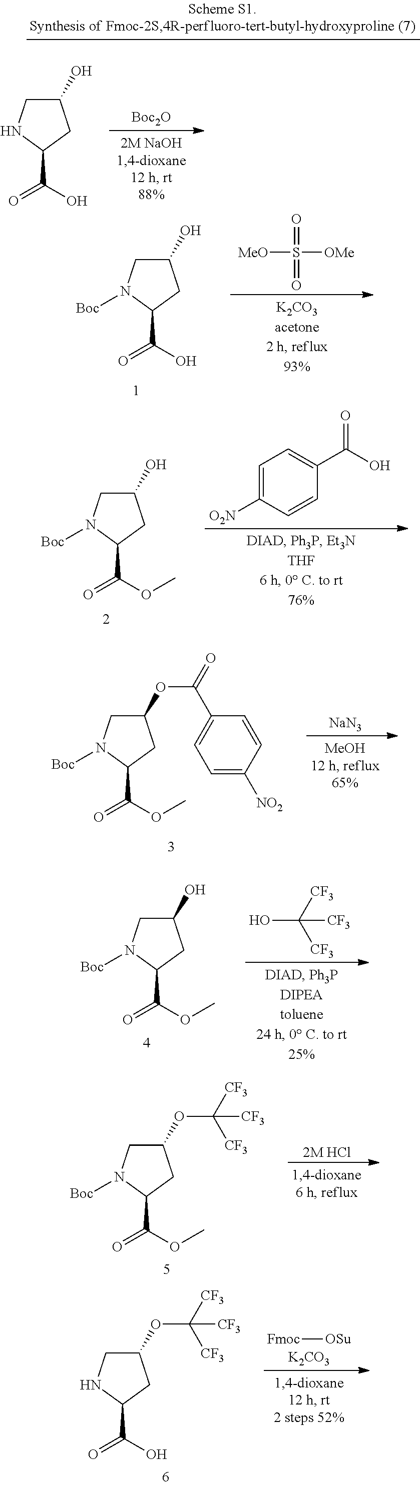

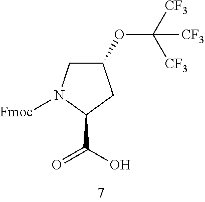

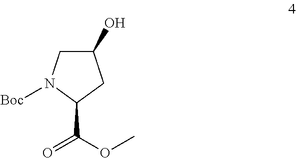 [SMILES: COC(=O)[C@@H]1C[C@H](O)CN1C(=O)OC(C)(C)C]

Boc-2S,4S-Hydroxyproline methyl ester (4). Compound 3 (2.0 g, 5.0 mmol) was dissolved in acetone (50 mL). Sodium azide (0.49 g, 7.5 mmol) was added and the solution was heated at reflux for 14 hours. The solution was allowed to cool to room temperature and solvent was removed under reduced pressure. The crude product was dissolved in ethyl acetate (50 mL) and washed with distilled water (2×50 mL). The solvent was removed and the crude product was redissolved in CH₂Cl₂ for purification. Compound 4 (0.80 g, 3.3 mmol) was purified via column chromatography in 2% methanol in CH₂Cl₂ to obtain a colorless oil in 65% yield. The NMR data corresponded to the literature values.

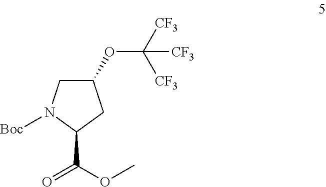 [SMILES: COC(=O)[C@@H]1C[C@@H](OC(C(F)(F)F)(C(F)(F)F)C(F)(F)F)CN1C(=O)OC(C)(C)C]

Boc-2S,4R-perfluoro-tert-butyl-hydroxyproline methyl ester (5). Compound 4 (2.23 g, 9.10 mmol) and Ph₃P (2.86 g, 10.9 mmol) were dissolved in toluene (91 mL). The reaction was conducted under a nitrogen atmosphere. The solution was cooled to 0° C. and stirred on ice for 10 minutes. DIAD (2.20 g, 2.15 mL, 10.9 mmol) was added dropwise to the solution over 15 minutes. Perfluoro-tert-butanol (4.30 g, 2.54 mL, 18.2 mmol) and DIPEA (2.35 g, 3.16 mL, 18.2 mmol) were added to the solution which was stirred on ice for another 5 minutes. The solution was removed from the ice bath, warmed to 45° C., and stirred for 24 hours. The solvent was removed under reduced pressure and the crude product was dissolved in ethyl acetate (50 mL). The crude product was washed with brine (2×75 mL) and dried over sodium sulfate. The solvent was removed under reduced pressure and crude product was redissolved in CH₂Cl₂ (50 mL). The crude product was purified via column chromatography (0-7% ethyl acetate in hexanes) to obtain compound 5 as a colorless oil in 25% yield. ¹H NMR (400 MHz, CDCl₃) δ 491 (S, 1H), 4.50-4.48 (m, 0.4H, cis), 4.40-4.37 (m, 0.6H, trans), 3.83-3.80 (dd, 0.6H, trans), 3.78-3.76 (dd, 0.4H, cis), 3.75 (s, 1.15H, cis), 3.75 (s, 1.84H, trans), 3.69-3.67 (d, J=12.5 Hz, 0.6H, trans), 3.60-3.58 (d, J=12.5 Hz, 0.4H, cis), 2.49-2.41 (m, 1H), 2.27-2.23 (m, 1H), 1.47 (s, 3.8H, cis), 1.42 (s, 5.21H, trans). ¹³C NMR (150.8 MHz, CDCl₃) δ 171.86, 152.36, 119.12 (q, J=293 Hz), 79.79, 77.09, 56.52, 51.47, 35.58, 27.16. ¹⁹F NMR (376.3 MHz, CDCl₃) δ −70.47 (trans conformation), −70.53 (cis conformation). HRMS expected: 463.1041, observed: 463.1051.

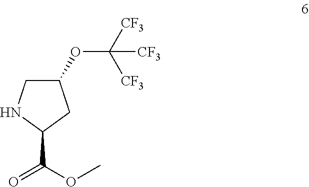 [SMILES: COC(=O)[C@@H]1C[C@@H](OC(C(F)(F)F)(C(F)(F)F)C(F)(F)F)CN1]

2S,4R-Perfluoro-tert-butyl-hydroxyproline (6). Compound 5 (1.3 g, 2.2 mmol) was dissolved in 1,4-dioxane (15 mL) and 4 M HCl (15 mL) was added. The solution was allowed to stir at reflux for 6 hours. Benzene was added (60 mL) and the water was removed as an azeotrope under reduced pressure. No purification was performed on compound 6 (0.78 g approximately). Compound 6 was used as a crude reagent without purification in the next step. ¹H NMR (D₂O) δ 5.27-5.25 (d, J=13.5 Hz, 1H), 4.50-4.47 (m, 1H), 3.73-3.59 (m, 2H), 2.75-2.65 (m, 1H), 2.50-2.36 (m, 1H). ¹⁹F NMR (376.3 MHz, D₂O) δ −70.43.

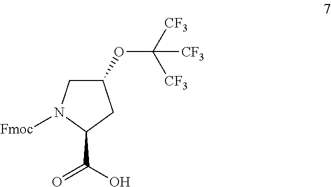

Fmoc-2S,4R-perfluoro-tert-butyl-hydroxyproline (7). Crude compound 6 (1.01 g, 2.90 mmol) was dissolved in 1,4-dioxane (15 mL) and H₂O (15 mL). Fmoc-OSu (1.17 g, 3.48 mmol) and K₂CO₃ (0.80 g, 5.80 mmol) were added and the solution was stirred for 14 hours at room temperature. The solvent was removed and crude product was acidified with 2 M HCl (10 mL). The crude product was extracted with ethyl acetate (2×20 mL). Compound 8 (0.86 g, 1.51 mmol) was purified via column chromatography (0-4% methanol in CH₂Cl₂) to obtain a white solid in 50% yield. ¹H (600 MHz, CDCl₃) δ 7.77-7.75 (d, 2H), 7.56-7.50 (m, 2H), 7.44-7.27 (m, 4H), 4.97 (m, 1H), 4.46-4.34 (m, 2H), 3.77 (m, 2H), 2.58-2.49 (m, 1H), 2.42-2.32 (m, 1H). ¹³C NMR (150.8 MHz, CDCl₃) δ 175.45, 155.34, 143.52, 141.33, 132.19, 128.57, 127.71, 124.93, 120.12 (q, J=293 Hz), 120.05, 117.21, 77.94, 68.26, 52.65, 47.01, 36.24, 28.24, 21.92. ¹⁹F NMR (564.5 MHz, CDCl₃) δ −70.38 (trans conformation), −70.43 (cis conformation). HRMS expected: 571.1041, observed: 571.1027.

### B. Synthesis of Fmoc-2S-4S-perfluoro-tert-butyl-hydroxyproline (10)

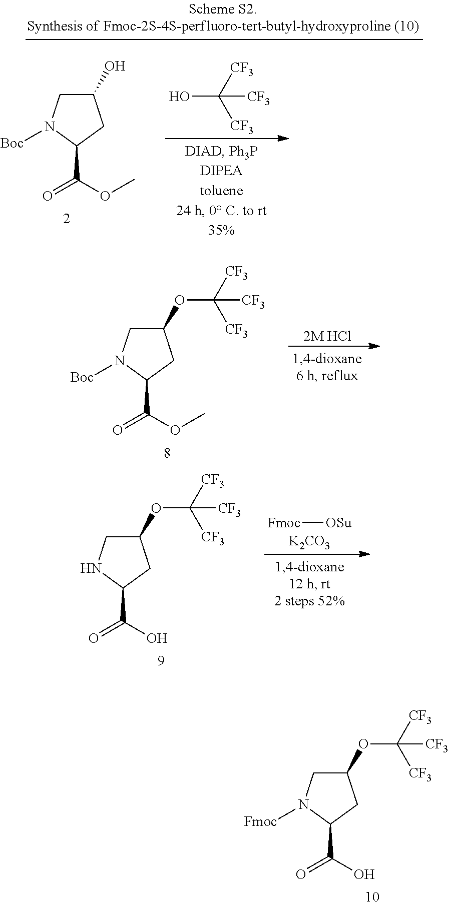

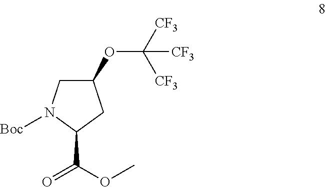 [SMILES: COC(=O)[C@@H]1C[C@H](OC(C(F)(F)F)(C(F)(F)F)C(F)(F)F)CN1C(=O)OC(C)(C)C]

Boc-2S,4S-perfluoro-tert-butyl-hydroxyproline methyl ester (8). Compound 2 (3.09 g, 12.6 mmol) and Ph₃P (3.96 g, 15.13 mmol) were dissolved in toluene (126 mL). The reaction was conducted under nitrogen atmosphere. The solution was cooled to 0° C. and stirred on ice for 10 minutes. DIAD (3.05 g, 2.98 mL, 15.1 mmol) was added dropwise to the solution over 15 minutes. Perfluoro-tert-butanol (5.95 g, 3.52 mL, 25.2 mmol) and DIPEA (3.18 g, 4.38 mL, 25.2 mmol) were added to the solution, which was stirred on ice for another 5 minutes. The solution was removed from the ice bath, warmed to 45° C., and stirred for 24 hours. The solvent was removed under reduced pressure and the crude product was dissolved in ethyl acetate (50 mL). The crude product was washed with brine (2×75 mL) and dried over sodium sulfate. The solvent was removed under reduced pressure and the crude product was redissolved in CH₂Cl₂ (50 mL). The crude product was purified via column chromatography (0-7% ethyl acetate in hexanes) to obtain compound 5 (2.07 g, 4.47 mmol) as a colorless oil in 36% yield. ¹H NMR (400 MHz, CDCl₃) 84.84 (s, 1H), 4.52-4.50 (m, 0.4H, trans), 4.41-4.38 (m, 0.6H, cis), 3.84-3.78 (dd, 0.6H, cis), 3.76-3.75 (dd, 0.4H, trans), 3.72, (s, 3H), 3.68-3.65 (d, J=12.95 Hz, 0.6H, cis), 3.58-3.55 (d, J=12.95 Hz, 0.4H, trans), 2.53-2.46 (m, 0.6H, cis), 2.44-2.38 (m, 0.4H, trans), 1.48 (s, 4H, trans), 1.43 (s, 5H, cis). ¹³C NMR (150.8 MHz, CDCl₃) δ 171.97, 153.50, 119.13 (q, J=293 Hz), 80.65, 78.61, 57.49, 52.63, 37.65, 28.24. ¹⁹F NMR (376.3 MHz, CDCl₃) δ −70.42 (trans conformation), −70.44 (cis conformation). HRMS expected: 463.1041 observed: 463.1042.

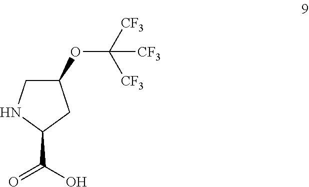 [SMILES: O=C(O)[C@@H]1C[C@H](OC(C(F)(F)F)(C(F)(F)F)C(F)(F)F)CN1]

2S,4S-perfluoro-tert-butyl-hydroxyproline (9). Compound 8 (2.0 g, 4.3 mmol) was dissolved in 1,4-dioxane (15 mL) and 4 M HCl (15 mL) was added. The solution was allowed to stir at reflux for 6 hours. Benzene was added (30 mL) and the water was removed as an azeotrophe under reduced pressure. No purification was performed on compound 9 (1.5 g approximately). Compound 9 was used as a crude reagent in the next step. ¹H NMR (D₂O) δ 5.25 (s, 0.4H, trans), 5.20 (s, 0.6H, cis), 4.44-4.42 (m, 1H), 3.72-3.68 (m, 1H). 3.64-3.62 (m, 0.4H), 3.58-3.55 (m, 0.6H), 2.70-2.55 (m, 21-1). ¹³C NMR (150.8 MHz, CDCl₃) δ 171.86, 152.36, 119.12 (q, J=293 Hz), 79.79, 77.09, 56.52, 51.47, 35.58, 27.16. ¹⁹F NMR (376.3 MHz, CDCl₃) δ −70.47 (trans conformation), −70.53 (cis conformation).

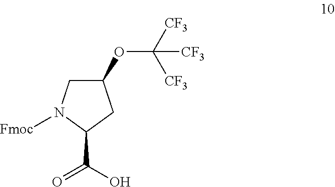

Fmoc-2S,4S-perfluoro-tert-butyl-hydroxyproline (10). Crude compound 9 (0.50 g, 1.4 mmol) was dissolved in 1,4-dioxane (7 mL). Fmoc-OSu (0.50 g, 1.4 mmol) and K₂CO₃ (0.39 g, 2.9 mmol) were added and the solution was stirred for 14 hours at room temperature. The solvent was removed and the crude product was acidified with 2 M HCl (10 mL). The crude product was extracted with ethyl acetate (2×20 mL). The crude mixture (0.40 g, 0.72 mmol) was purified via silica column chromatography (0-4% methanol in CH₂Cl₂) to obtain compound 10 as a white solid in 50% yield. ¹H (600 MHz, CDCl₃) δ 7.77-7.69 (d, 2H), 7.60-7.51 (m, 2H), 7.42-7.28 (m, 4H), 4.88 (m, 1H), 4.61-4.15 (m, 4H), 3.84-3.68 (m, 2H), 2.57-2.41 (m, 2H), ¹³C NMR (150.8 MHz, CDCl₃) δ 175.65, 143.59, 141.39, 127.78, 127.08, 124.78, 120.00 (J=293 Hz), 117.20, 78.22, 67.81, 57.33, 52.89, 47.10, 36.49, 21.87. ¹⁹F NMR (564.5 MHz, CDCl₃) δ −70.42 (trans conformation), −70.50 (cis conformation). HRMS expected: 571.1041, observed: 571.1038.

### C. Synthesis of Fmoc-Perfluoro-Tert-Butyl-Homoserine

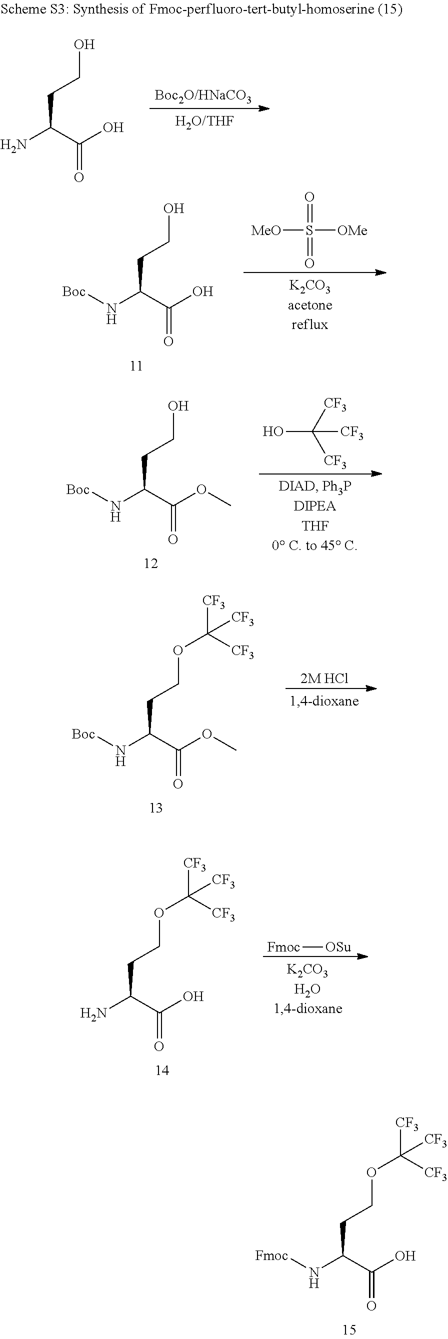

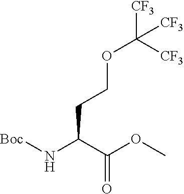 [SMILES: COC(=O)[C@H](CCOC(C(F)(F)F)(C(F)(F)F)C(F)(F)F)NC(=O)OC(C)(C)C]

Boc-perfluoro-tert-butyl-homoserine methyl ester (13). Compound 12 (1.67 g, 7.17 mmol) and Ph₃P (2.82 g, 10.75 mmol) were dissolved in 50 mL THF in a three-neck round bottom flask with a condenser. The reaction was performed under nitrogen atmosphere. The solution was cooled to 0° C. before DIAD (2.17 g, 2.11 mL, 10.75 mmol) was added dropwise over 15 minutes. The reaction was allowed to stir on ice for 5 minutes before the dropwise addition of the perfluoro-tert-butanol (3.39 g, 2.00 mL, 14.34 mmol) over 5 minutes. The reaction mixture was allowed to stir on ice for another 5 minutes before heating to 50° C. for 24 hours. Upon completion of the reaction, solvent was removed under reduced pressure. The crude reaction mixture was purified via column chromatography (0 to 10% ethyl acetate in hexanes) to obtain compound 13 as a white solid in 54% yield. ¹H (600 MHz, CDCl₃) δ 5.17 (s, 1H), 4.41 (s, 1H), 4.13 (d, 2H), 3.75 (s, 3H), 2.31-2.27 (m, 1H), 2.17-2.10 (m, 1H), 1.44 (s, 9H). ¹³C NMR (150.8 MHz, CDCl₃) δ 172.10, 155.23, 119.13 (q, J=293 Hz), 79.66, 66.05, 51.91, 50.32, 31.89, 27.77. ¹⁹F NMR (376.3 MHz, CDCl₃) δ −70.9.

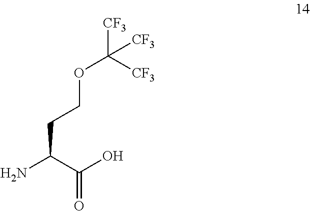 [SMILES: N[C@@H](CCOC(C(F)(F)F)(C(F)(F)F)C(F)(F)F)C(=O)O]

Perfluoro-tert-butyl-homoserine (14). Compound 13 (1.74 g, 3.86 mmol) and LiOH (0.231 g, 9.65 mmol) were dissolved in 35 mL THF and 15 mL H₂O. The reaction was allowed to stir at room temperature for 12 hours. The reaction mixture was acidified to pH 2 using HCl, followed by removal of the THF under reduced pressure. The remaining mixture was extracted with ethyl acetate (3×20 mL). The organic layers were collected and concentrated under reduced pressure as crude Boc-perfluoro-tert-butyl-homoserine. The product was redissolved in 10 mL 2M HCl and 10 mL 1,4-dioxane. The reaction was allowed to stir for 6 hours. The solvent was removed under reduced pressure. No purification was performed on compound 14 (1.30 g approximately). Compound 14 was used as a crude reagent in the next step. ¹H (600 MHz, D₂O) δ 4.33-4.30 (m, 2H), 4.21-4.18 (m, 1H), 2.40-2.30 (m, 2H). ¹³C NMR (150.8 MHz, D₂O) δ 171.20, 119.13 (q, J=293 Hz), 65.82, 50.07, 30.09. ¹⁹F NMR (376.3 MHz, D₂O) δ −70.60,

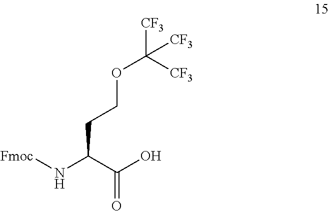

Fmoc-perfluoro-tert-butyl-homoserine (15). Compound 14 (approximately 1.30 g) was dissolved in 20 mL H₂O and 20 mL 1,4-dioxane. Fmoc-OSu (1.69 g, 5.02 mmol) and K₂CO₃ (0.80 g, 5.79 mmol) were added and the reaction was stirred for 14 hours at room temperature. The reaction mixture was acidified to pH 1 and extracted with ethyl acetate (3×20 mL). The organic layers were combined and the solvent was removed under reduced pressure. The crude reaction mixture was dissolved in CH₂Cl₂ and purified via column chromatography (0 to 2% methanol in CH₂Cl₂). Compound 15 was obtained as a white solid in 48% yield. ¹H (600 MHz, CDCl₃) δ 7.76 (d, 2H), 7.56 (dd, 2H), 7.40 (dd, 2H), 7.31 (dd, 2H), 5.45 (d, 1H), 4.51 (d, 1H), 4.42 (d, 2H), 4.22 (dd, 1H), 4.16 (s, 1H), 2.37-2.32 (m, 1H), 2.30-2.25 (m, 1H), ¹³C NMR (150.8 MHz, CDCl₃) δ 143.58, 141.36, 127.79, 127.07, 125.01, 120.02, 67.26, 66.03, 50.84, 47.11, 31.43. ¹⁹F NMR (376.3 MHz, CDCl₃) δ −70.28.

### D. Synthesis of Perfluoro-Tert-Butylalanine

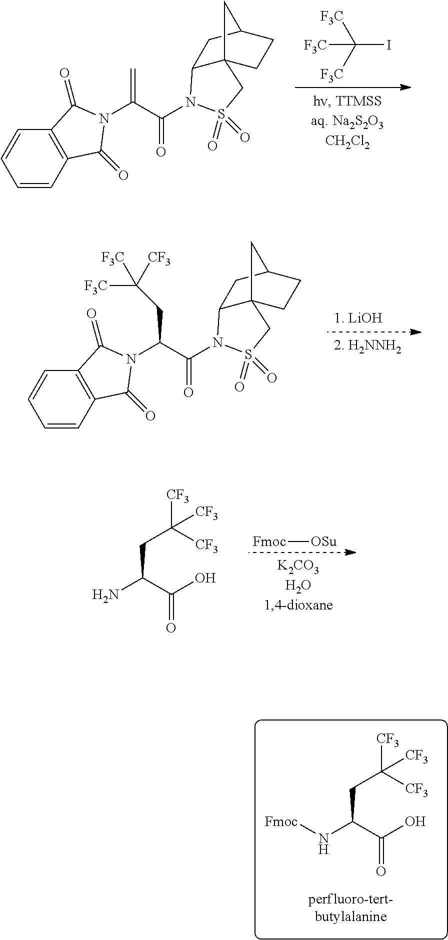

### E. Synthesis of Perfluoro-Tert-Butyltyrosine

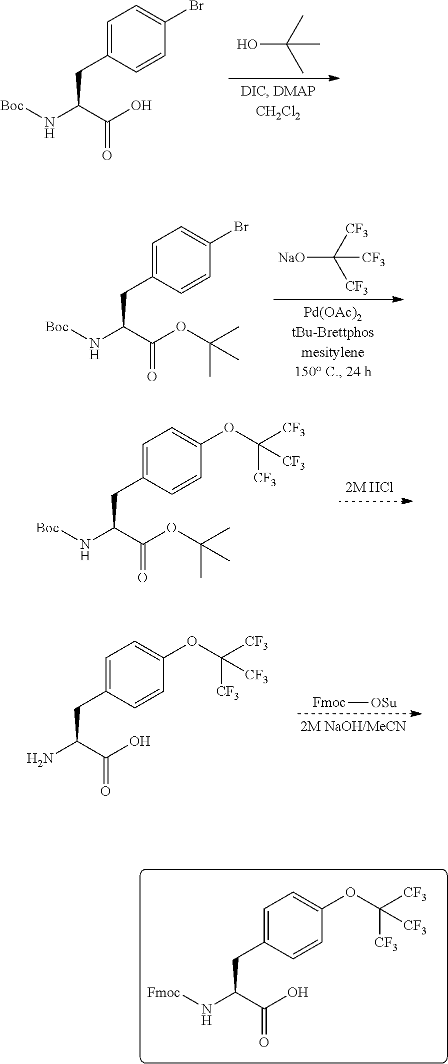

### F. Synthesis of Perfluoro-Tert-Butyl Glycine

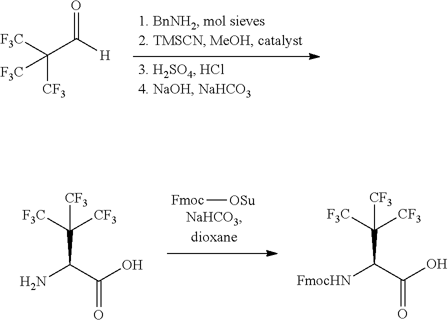

### G. Synthesis of Perfluoro-Tert-Butyl Aspartate and Glutamate

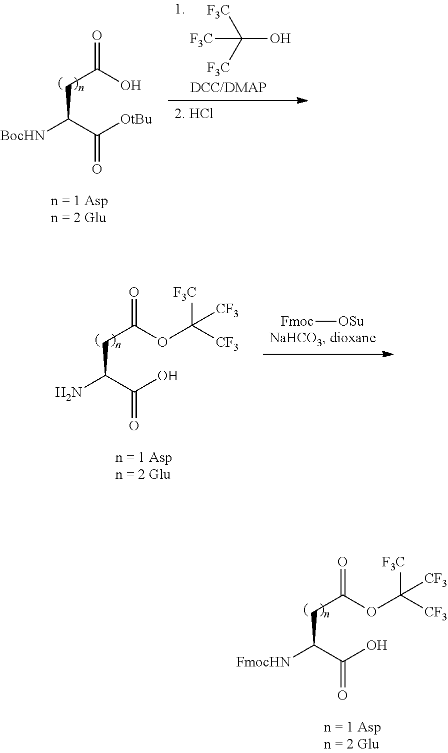

### Example 2

### Synthesis of Peptides Containing Perfluoro-Tert-Butyl Hydroxyproline ((2S,4R)-L-Hyp(OC(CF₃)₃ and (2S,4S)-L-hyp(OC(CF₃)₃)

The application of the Mitsunobu reaction with perfluoro-tert-butanol (pK, 5) to incorporate a perfluoro-tert-butyl ether with either stereochemistry (55, 56) has not been reported so far.⁴⁴ tert-Butyl groups have broad applications in medicinal chemistry and catalysis due to their sterics, hydrophobicity, and symmetry, which permits target binding with a reduced cost in conformational entropy. tert-Butyl groups have similar advantages in amino acids and peptides, with tert-leucine observed in pharmaceuticals and catalysts due to a strong conformational bias and steric effect.⁴⁵ A perfluoro-tert-butyl group would be expected to have enhanced steric and hydrophobic effects over a tert-butyl group, while also introducing nine equivalent fluorines that would provide a strong singlet signal in ¹⁹F NMR, suggesting its possible use as a functional probe.^{1e, 1l, m, 44b, 46} Mitsunobu reactions of perfluoro-tert-butanol proceeded with good conversion on solid phase to generate both 4R- and 4S-perfluoro-tert-butylhydroxyproline ethers (55, 56) within peptides.

The ¹⁹F NMR spectra corroborated the K_{trans/cis} values observed by ¹H NMR. The perfluoro-tert-butyl hydroxyproline peptides were noteworthy for the fluorine signal existing as a sharp singlet, with 9 equivalent fluorines, suggesting potential applications as a magnetic resonance probe with high signal to noise.^{44b, 46} Automated synthesis of peptide Ac-TYHypN—NH₂ (1) via trityl hydroxyl protection Scheme S4. Proline editing general approach: automated synthesis of the peptide Ac-TYHypN—NH₂ (1) via trityl hydroxyl protection.^{a}

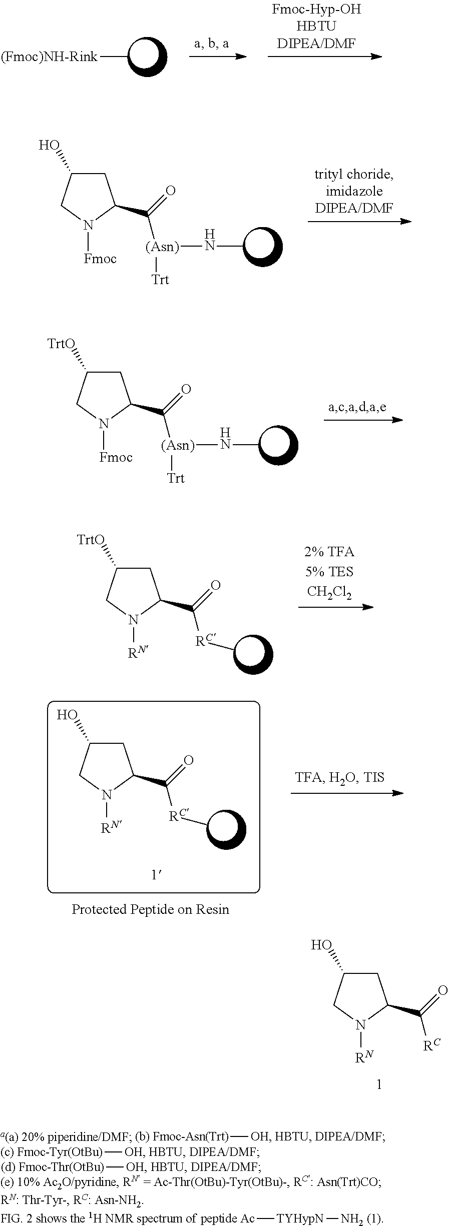

The peptide Ac-TYHypN—NH₂ (1) was synthesized via standard Fmoc solid phase peptide synthesis with Rink amide resin (0.25 mmol) (Scheme S4). The resin was swelled in DMF (5 min) prior to the start of the synthesis. Amino acid couplings were performed using Fmoc amino acids (1 mmol, 4 equiv) and HBTU (1 mmol, 4 equiv). The following steps were used for each cycle of peptide synthesis: (1) removal of the Fmoc group (20% piperidine in DMF, 2×5 min); (2) resin wash (DMF, 5×1 min); (3) amide coupling (amino acid, HBTU, 0.05 M DIPEA in DMF, 50 min); (4) resin wash (DMF, 3×1 min). After addition of the final residue, the N-terminal Fmoc group was removed (20% piperidine in DMF, 3×5 min) and the amino terminus acetylated (10% acetic anhydride in pyridine, 5 min). The resin was washed with DMF (6×) and Cl₂Cl₂ (3×). Coupling and in situ protection of hydroxyproline with a trityl protecting group

The coupling of hydroxyproline was conducted for 60 min, followed by two cycles of in situ protection of the hydroxyl group by the trityl group (Scheme S4). In each cycle, trityl chloride (697 mg, 2.5 mmol) and imidazole (170 mg, 2.5 mmol) were dissolved in anhydrous CH₂Cl₂ (2.0 mL) and mixed with standard activator solution (8% DIPEA in DMF) and coupled for 60 min. The protection steps were performed in an automated manner by programming the protection steps as a triple coupling in the peptide synthesis (first step, amide bond formation; second and third steps, trityl protection). The peptide synthesis was completed as described in Scheme S4.

### Deprotection of Trityl Group of Ac-TYP(4R—O(Trt))N—NH₂

The trityl group was selectively removed by addition of 2% TFA, 5% TES in CH₂Cl₂ to the resin and mixed for 1 min (Scheme S4). The solution was immediately removed via filtration on a water aspirator. This process was repeated twice. The resin was washed with CH₂Cl₂(3×) and CH₃OH (2×) and dried with diethyl ether.

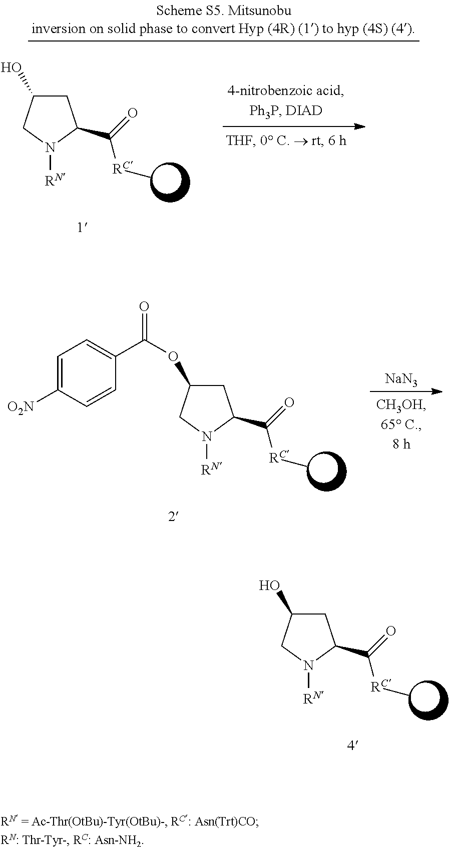

To triphenylphosphine (Ph₃P) (262 mg, 1.0 mmol) dissolved in THF (2.0 mL) at 0° C. was added diisopropylazodicarboxylate (DIAD) (197 μL, 1.0 mmol). Resin-bound peptide 1′ was then added to the reaction mixture followed by p-nitrobenzoic acid (94.0 mg, 1.0 mmol). The reaction mixture was allowed to react with gentle mixing at room temperature for 12 h to obtain peptide 2′. The chemically modified peptide on resin was then filtered and washed with DMF (3×) and CH₂Cl₂ (3×) on a water aspirator.

The deprotection of the hydroxyl group was performed by gentle stirring of the peptide 2′ with a solution of sodium azide (NaN₃) (65.0 mg, 1.0 mmol) in CH₃OH (2.0 mL) at 65° C. for 12 h. The chemically modified peptide 4′ was then filtered and washed with DMF (3×) and CHCl₂ (3×) on a water aspirator.

FIG. 3 shows the ¹H NMR spectrum of peptide Ac-TYhypN—NH₂ (4).

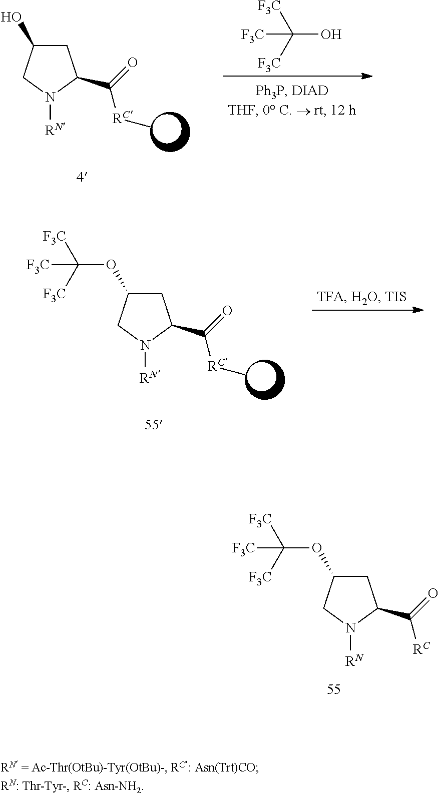

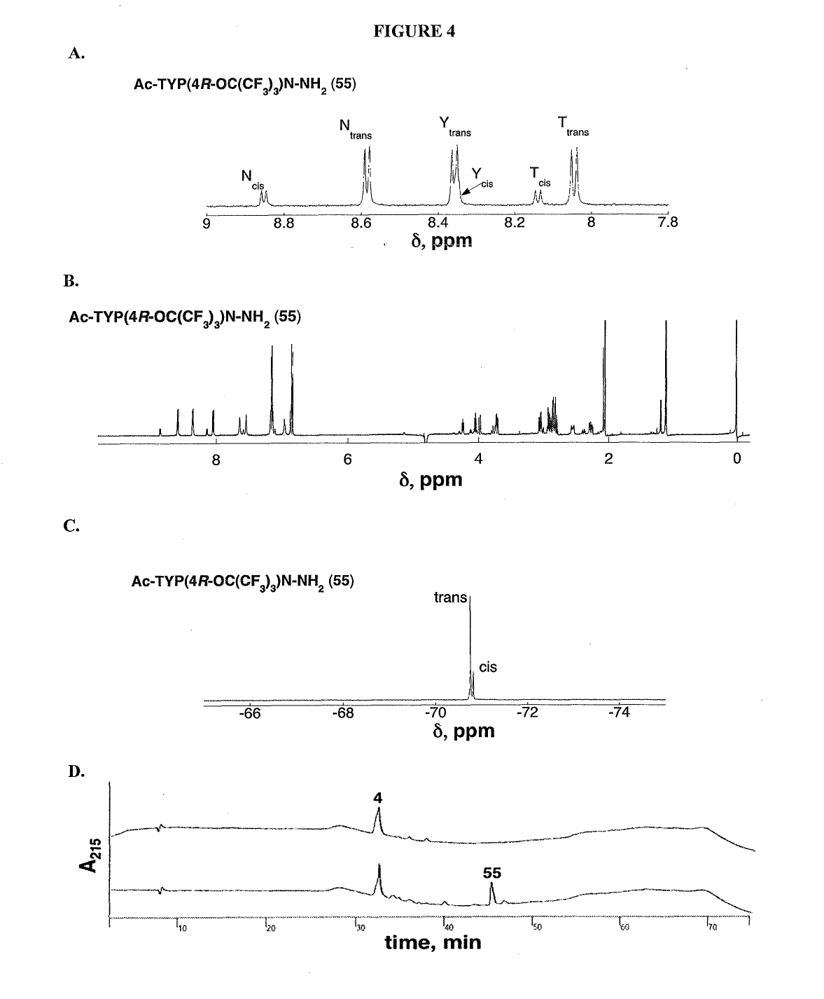

To Ph₃P (65.6 mg, 0.25 mmol) dissolved in anhydrous THF (1.0 mL) at 0° C. was added DIAD (49.2 μL, 0.25 mmol). Resin-bound peptide 4′ was then added to the reaction mixture followed by perfluoro-tert-butanol (35.0 μL, 0.25 mmol). The reaction mixture was allowed to react with gentle mixing at room temperature for 12 h. The modified peptide was then subjected to general procedure A to obtain peptide 55: expected mass 768.1, observed mass (M+Na) 791.1, t_{R}=53.6 min, ¹⁹F NMR (376.5 MHz): 6=−70.7 (s, Fₜᵣₐₙₛ), −70.8 (s, F_{cis}). FIG. 4 shows the ¹H NMR spectrum of peptide Ac-TYP(4R—OC(CF₃)₃)N—NH₂ (55), the ¹⁹F NMR spectrum of peptide Ac-TYP(4R—OC(CF₃)₃)N—NH₂ (55), and the Crude HPLC chromatogram.

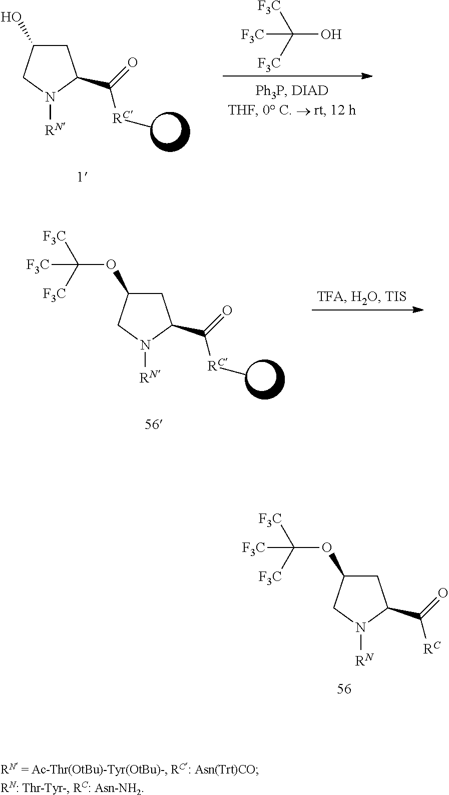

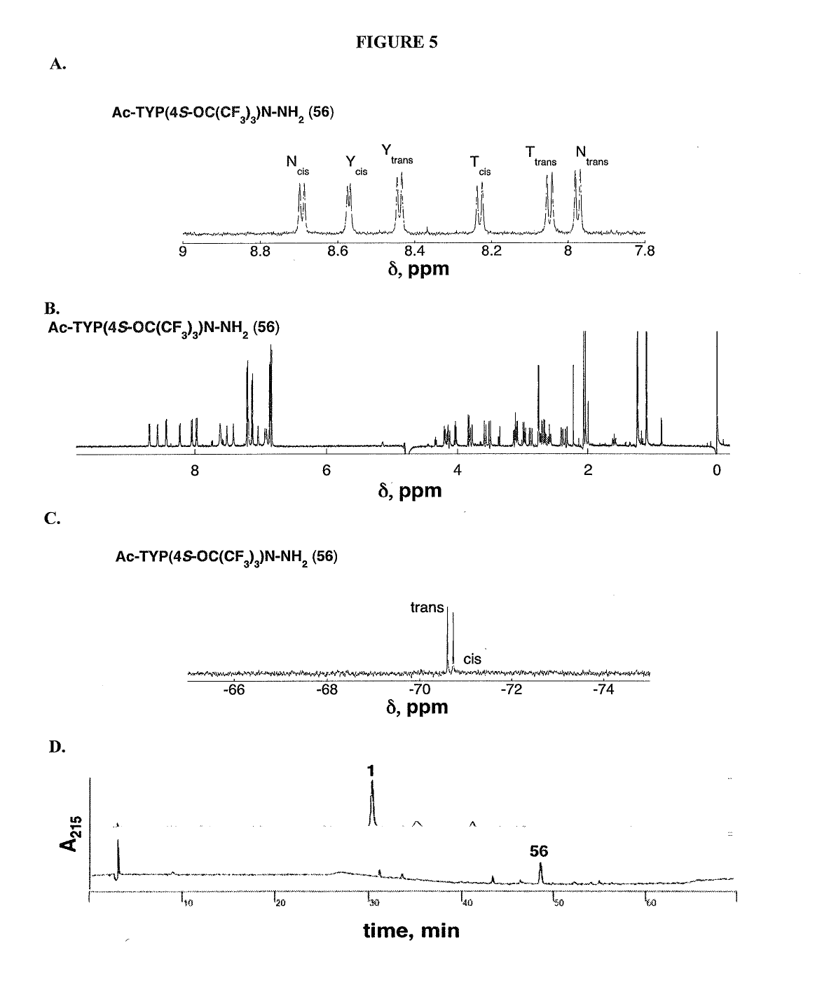

To Ph₃P (65.6 mg, 0.25 mmol) dissolved in anhydrous THF (1.0 mL) at 0° C. was added DIAD (49.2 μL, 0.25 mmol). Resin-bound peptide 1′ was then added to the reaction mixture followed by perfluoro-tert-butanol (35.0 μL, 0.25 mmol). The reaction mixture was allowed to react with gentle mixing at room temperature for 12 h. The modified peptide was then subjected to general procedure A to obtain peptide 56: expected mass 768.1, observed mass (M+Na) 791.1, t_{R}=51.3 min. ¹⁹F NMR (376.5 MHz): 6=−70.6 (s, Fₜᵣₐₙₛ), −70.7 (s, F_{cis}). FIG. 5 shows the ¹H NMR spectrum of peptide Ac-TYP(4S—OC(CF₃)₃)N—NH₂ (56), the ¹⁹F NMR spectrum of peptide Ac-TYP(4S—OC(CF₃)₃)N—NH₂ (56), and crude HPLC chromatogram.

### REFERENCES

(1) (e) Yoder, N. C.; Kumar, K. Chem. Soc. Rev. 2002, 31, 335. (l) Merkel, L.; Budisa, N. Org. Biomol. Chem. 2012, 10, 7241; (m) Salwiczek, M.; Nyakatura, E. K.; Gerling, U. I. M.; Ye, S. J.; Koksch, B. Chem. Soc. Rev. 2012, 41, 2135.
(11) Shoulders, M. D.; Hodges, J. A.; Raines, R. T. J Am. Chem. Soc. 2006, 128, 8112.
(44) (a) Sebesta, D. P.; Orourke, S. S.; Pieken, W. A. J. Org. Chem. 1996, 61, 361; (b) Jiang, Z. X.; Yu, Y. B. J. Org Chem. 2007, 72, 1464.
(45) (a) Bisel, P.; Al-Momani, L.; Muller, M. Org. Biomol. Chem. 2008, 6, 2655; (b) Lyu, P. C.; Sherman, J. C.; Chen, A.; Kallenbach, N. R. Proc. Natl. Acad. Sci. USA 1991, 88, 5317; (c) Cornish, V. W.; Kaplan, M. I.; Veenstra, D. L.; Kollman, P. A.; Schultz, P. G. Biochemistry 1994, 33, 12022; (d) Sigman, M. S.; Jacobsen, E. N. J. Am. Chem. Soc. 1998, 120, 4901; (e) Guerin, D. J.; Miller, S. J. J. Am. Chem. Soc. 2002, 124, 2134; (f) Formaggio, F.; Baldini, C.; Moretto, V.; Crisma, M.; Kaptein, B.; Broxterman, Q. B.; Toniolo, C. Chem. Eur. J. 2005, 11, 2395; (g) Bielska, A. A.; Zondlo, N. J. Biochemistry 2006, 45, 5527; (h) Brown, A. M.; Zondlo, N. J. Biochemistry 2012, 51, 5041.
(46) (a) Dalvit, C. Prog. Nucl. Magn. Reson. Spectrosc. 2007, 51, 243; (b) Papeo, G.; Giordano, P.; Brasca, M. G.; Buzzo, F.; Caronni, D.; Ciprandi, F.; Mongelli, N.; Veronesi, M.; Vulpetti, A.; Dalvit, C. J. Am. Chem. Soc. 2007, 129, 5665.

### Example 3

### 2S,4R-Perfluoro-Tert-Butyl-Hydroxyproline Containing Kemptide Phosphorylation by PKA

cAMP-dependent Protein Kinase catalytic subunit (PKA) was purchased from New England BioLabs (Cat. #P6000S). Reaction mixtures were prepared to a final volume of 25 μL as follows: 50 mM Tris-HCl (pH 7.5), 10 mM MgCl₂, 2 mM DTT, 100 μM nonphosphorylated peptide, 40 μM ATP, and 1 μL enzyme solution (2,500 units). After incubation at 37° C. for 5 mins, the reaction mixture was heated to 100° C. for 15 minutes to inactivate the enzyme. The solution was then centrifuged for 30 seconds and diluted with 425 μL autoclaved water and 50 μL D₂O before transferring to an NMR tube. After NMR, the solution was injected on the HPLC to verify NMR results.

### Real Time NMR of 2S,4R-Perfluoro-Tert-Butyl-Hydroxyproline Containing Kemptide Phosphorylation by PKA

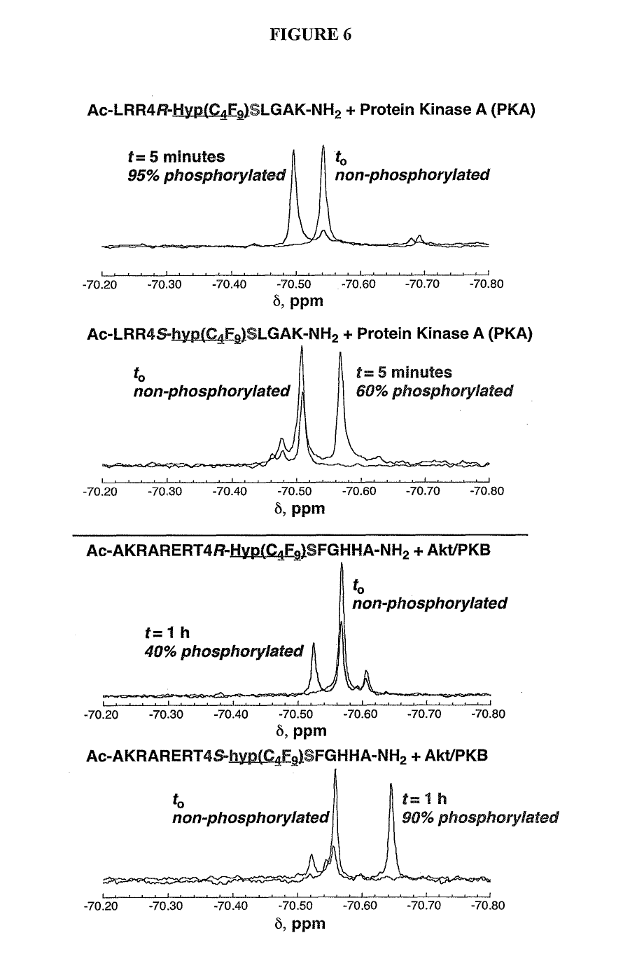

PKA (1 μL, New England BioLabs) was diluted in buffer containing 50 mM Tris-HCl (pH 7.5) and 10 mM MgCl₂ (39 μL). Reaction mixtures were prepared to a final volume of 500 μL as follows: 10 μM nonphosphorylated peptide, 80 μM ATP, 50 mM Tris-HCl (pH 7.5), 10 mM MgCl₂, 5 mM DTT, and 10% D₂O. Experiments were conducted at 310 K. One NMR experiment was taken before enzyme was added as t=0. The tube was removed from the NMR and 5 μL (313 U) of the diluted enzyme was added. A total of six more experiments were completed without removing the sample from the NMR. Each experiment was performed using a ¹⁹F coupled method with 128 scans with a 10 ppm sweep width and a relaxation delay of 1.5 seconds. Each experiment was 5 mins and 14 seconds for a total time of 31.4 mins. After the last experiment, the sample was injected on the HPLC to verify NMR results. FIG. 6 shows phosphorylation of the peptides.

### Cell Culture and Cell Extracts

HeLa cells were cultured at 37° C. humidified environment containing 5% CO₂ with Dulbecco's Modified Eagle Medium (DMEM) with 10% heat inactivated fetal bovine serum (FBS), L-Glutamine (2 mM), penicillin (100 units/mL), and streptomycin (100 μg/mL). Twenty hours before lysate preparation, cells were starved with DMEM containing 0.5% FBS, The media was removed and the cells were washed with 4 mL 1×DPBS. The cells were trypsinized and centrifuged (3.5 rpm, 1 min). The pellet was resuspended in 2 mL 1×DPBS and centrifuged (3.5 rpm, 1 min). The pellet was resuspended in 1 mL Buffer A (0.4 M HEPES (pH 7.9), 60 mM MgCl₂, 400 mM KCl, 20 mM DTT, and 8 mM PMSF) and centrifuged (3.5 rpm, 1 min). The pellet was resuspended in Buffer A and incubated on ice for 10 minutes. The solution was then vortexed for 30 second, centrifuged (3.5 rpm, 1 min), and resuspended in Buffer B (100 mM HEPES (pH 7.9), 4.1 M NaCl, 14.7 mM MgCl₂, 200 uM EDTA, 5 mM DTT, 5 mM PMSF, and 2.5% gylcerol). The solution was incubated on ice for 15 minutes and then centrifuged (3.5 rpm, 5 min). The supernatant was divided into aliquots and frozen at −80° C.

### Real Time NMR of 2S,4R-Perfluoro-Tert-Butyl-Hydroxyproline Containing Kemptide Phosphorylation by HeLa Cell Lysates

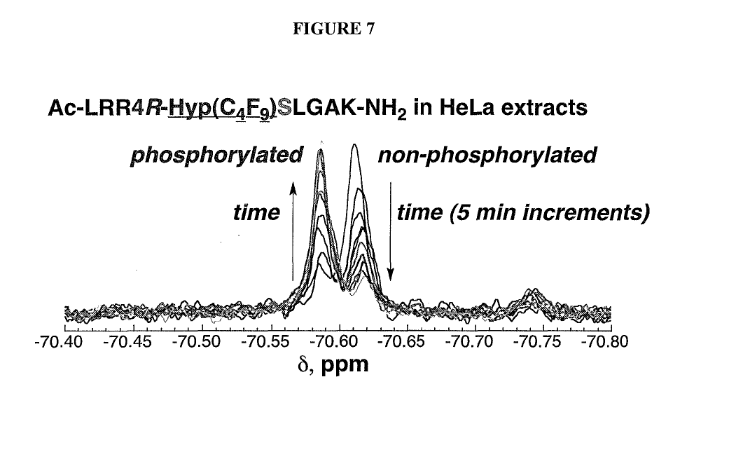

The reaction mixtures of peptide in HeLa cell extracts were prepared to a final volume of 500 μL as follows: stock solutions were mixed to yield final concentrations of 160 μM ATP, 200 μM β-glycerophosphate, 200 μM sodium orthovanadate (Na₃VO₄), 50 mM Tris-HCl, 10 mM MgCl₂, 20 μM non-phosphorylated peptide, 10% D₂O and 175 μL cell extracts. Experiments were conducted at 310 K. Peptide, ATP, and inhibitors, were dissolved in D₂O and buffer. The cell lysates were added and the entire reaction mixture was transfer to an NMR tube. A total of twelve experiments were completed, removing the sample after the first six to supplement ATP (80 μM, 10 μL). NMR experiments were run as per real time in vitro assays for a total of 62.8 mins. Reactions were then diluted with 500 μL of distilled water and injected on the HPLC to verify NMR results. FIG. 7 shows real time detection of PKA activity and PKA inhibition.

Various terms relating to the systems, methods, and other aspects of the present invention are used throughout the specification and claims. Such terms are to be given their ordinary meaning in the art unless otherwise indicated.

All documents, books, manuals, papers, patents, published patent applications, guides, abstracts, and/or other references cited herein are incorporated by reference in their entireties. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1: An analogue of an alpha amino acid, comprising a perfluoro-tert-butyl group.

2: The analogue of claim 1, wherein the analogue is selected from the group consisting of perfluoro-tert-butyl hydroxyproline, perfluoro-tert-butylalanine, perfluoro-tert-butyl homoserine, perfluoro-tert-butyl glycine, perfluoro-tert-butyl aspartate, perfluoro-tert-butyl glutamate and perfluoro-tert-butyl tyrosine.

3: The analogue of claim 1, wherein the analogue is perfluoro-tert-butyl hydroxyproline (Hyp).

4: The analogue of claim 3, wherein the Hyp is selected from the group consisting of 2S,4R (Hyp), 2S,4S (Hyp), 2R,4R (Hyp) and 2R,4R (Hyp).

5: The analogue of claim 1, wherein the analogue is Fmoc perfluoro-tert-butyl hydroxyproline or Boc-perfluoro-tert-butyl hydroxyproline.

6: A molecule comprising the analogue of claim 1.

7: A composition comprising the molecule of claim 6.

8: A method for diagnosing, treating or preventing a disease or condition in a subject in need thereof, comprising administrating to the subject an effective amount of the composition of claim 7.

9: A method for detecting a target molecule in a sample, comprising
(a) exposing the sample to a test molecule comprising the analogue of claim 1, wherein the analogue interacts with the target molecule, and
(b) detecting the interaction, wherein the presence of the interaction indicates the presence of the target molecule in the sample.

10: The method of claim 9, further comprising quantifying the target molecule in the sample.

11: The method of claim 9, wherein the target molecule is modified upon exposure, further comprising
(c) detecting the modified target molecule, wherein the modification of the target molecule indicates the presence of the interaction.

12: The method of claim 11, wherein the affinity between the target molecule and a subject molecule in the sample is altered upon exposure.

13: The method of claim 9, wherein the test molecule is modified upon exposure, further comprising
(c) detecting the modified test molecule, wherein the modification of the test molecule indicates the presence of the interaction.

14: The method of claim 9, wherein the test molecule binds a biological molecule in the sample upon exposure, further comprising
(c) detecting the test molecule bound to the biological molecule, wherein the presence of the test molecule bound to the biological molecule indicates the presence of the interaction.

15: The method of claim 9, wherein the test molecule binds a cell in the sample upon exposure, further comprising
(c) detecting the test molecule bound the biological molecule, wherein the presence of the test molecule bound to the cell indicates the presence of the interaction.

16: The method of claim 13, wherein the test molecule is detected by ¹⁹F NMR spectroscopy, magnetic resonance stimulation (MRS) or magnetic resonance imaging (MRI).

17: The method of claim 16, wherein the test molecule is detected by magnetic resonance imaging (MRI) in vivo.

18: The method of claim 9, wherein the sample is obtained from a subject or in a subject, wherein the presence of the target molecule in the sample indicates that the subject suffers or is predisposed to a disease or condition associated with the target molecule.

19: The method of claim 18, further comprising treating or preventing the disease or condition in the subject.

20: A method for modifying a target molecule in a sample, comprising exposing the sample to an effective amount of a test molecule comprising the analogue of claim 1.


SEQUENCE LISTING (8 sequences)

SEQ ID NO: 1 - protein, 4 aa, Artificial Sequence
  Synthetic
TYXN

SEQ ID NO: 2 - protein, 4 aa, Artificial Sequence
  Synthetic
TYXN

SEQ ID NO: 3 - protein, 4 aa, Artificial Sequence
  Synthetic
TYXN

SEQ ID NO: 4 - protein, 4 aa, Artificial Sequence
  Synthetic
TYXN

SEQ ID NO: 5 - protein, 9 aa, Artificial Sequence
  Synthetic
LRRXSLGAK

SEQ ID NO: 6 - protein, 9 aa, Artificial Sequence
  Synthetic
LRRXSLGAK

SEQ ID NO: 7 - protein, 15 aa, Artificial Sequence
  Synthetic
AKRARERTXSFGHHA

SEQ ID NO: 8 - protein, 15 aa, Artificial Sequence
  Synttheic
AKRARERTXSFGHHA